(19) United States
(12) Patent Application Publication
(10) Pub. No.: US 2005/0222650 A1
(43) Pub. Date: Oct. 6, 2005

(54) Neurosurgical device for thermally affecting tissue having invertable lumen

(75) Inventor: John M. Kirkman JR., Kingston, NH (US)

Correspondence Address: CHRISTOPHER & WEISBERG, P.A., 200 EAST LAS OLAS BOULEVARD, SUITE 2040, FORT LAUDERDALE, FL, 33301, US

(73) Assignee: Seacoast Technologies, Inc.

(21) Appl. No.: 10/818,231

(22) Filed: Apr. 5, 2004

(51) Int. Cl. A61F 7/00

(52) U.S. Cl. 607/96

(57) ABSTRACT

A device and method are provided for effecting a thermal change within a selection region of a skull, such as a particular surface portion of the brain, the tissue that encloses the brain or the fluid within the skull. A first elongate, flexible member having a proximal end, a distal end, and a wall portion having an outer surface and an inner surface is provided. The wall portion defines a lumen extending from the proximal end to the distal end, wherein the proximal end is open and the distal end is closed. The wall portion further defines a passage from the outer surface to the inner surface at a point between the proximal end and the distal end. A second elongate, flexible member having a proximal end, a distal end, and a wall portion having an outer surface and an inner surface is also provided, the wall portion defining a lumen extending from the proximal end to the distal end, wherein the proximal end is open and the distal end is closed, and wherein the distal end is secured to the first elongate, flexible member between the passage and the distal end of the first elongate, flexible member.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a medical device and to a method of treatment, and more particularly to a device and method for thermal treatment of selected regions within the cranium, such as the surface of the brain, as well as other areas of the body, such as the spinal cord.

### BACKGROUND OF THE INVENTION

It has long been known that thermally treating living tissue can produce therapeutic effects. However, some areas of the body are easier to treat than others. For example, thermal treatment within the cranium or skull, such as brain tissue, is particularly difficult and such treatment has largely been attempted using devices that are placed on external regions of the patient, such as the head or neck, or devices that attempt to cool the brain by cooling its blood supply.

The former method of treatment, such as through the use of cooling helmets or neck collars, does not impart a change deep enough inside the patient's skull to effectively render treatment in the case of brain trauma. In another method, inserting a chilled catheter into a major artery leading to the brain cools the brain's blood supply. However, the resultant cooling is not sufficiently bounded to allow for localized cooling to specific tissue areas of the brain, such as a small surface portion, and is impractical due to a number of shortcomings that can be injurious to the patient. Such shortcomings include the introduction of cardiac arrhythmias, suppression of immune function and coagulopathies.

Only recently have devices been proposed to treat brain tissue and the like, in which the devices are placed in direct contact with the tissue to be treated. Direct contact allows localized, controlled cooling of the specific tissue (or fluid) to be treated. Such devices are typically placed in contact with the tissue to be treated by performing a craniotomy or creating a burr hole in the skull. It is preferable to create as small an opening in the skull as possible to minimize infection, facilitate healing and reduce the likelihood of other detrimental medical complications.

Once the opening in the skull is created, the device is inserted through the opening and treatment commenced. The inserted device may be an inflatable device, which uses a circulating thermally conductive fluid to impart the thermal change to the tissue. Once inflated for treatment, if the opening in the skull is kept to a minimum, the device may be expanded to a size larger than the initial opening.

Once treatment is completed, the device must be removed. Even if the device is deflated, there is a good chance that the removal back through the opening will require dragging at least a portion of the device across the tissue. This dragging may result in further damage to the very tissue that was treated. It is therefore desirable to have a device which can be used to thermally treat tissue by placement in direct contact therewith, in which the device can be placed through as small an opening in the body as possible, and which is removable in a manner which does not result in the dragging of the device across the tissue.

### SUMMARY OF THE INVENTION

The present invention advantageously provides a method and device for effecting a thermal change within a selection region of a skull, such as a particular surface portion of the brain, the tissue that encloses the brain, or the fluid within the skull. In an exemplary device of the invention, a first elongate, flexible member having a proximal end, a distal end, and a wall portion having an outer surface and an inner surface is provided. The wall portion defines a lumen extending from the proximal end to the distal end, wherein the proximal end is open and the distal end is closed. The wall portion further defines a passage from the outer surface to the inner surface at a point between the proximal end and the distal end. A second elongate, flexible member having a proximal end, a distal end, and a wall portion having an outer surface and an inner surface is also provided, the wall portion defining a lumen extending from the proximal end to the distal end, wherein the proximal end is open and the distal end is closed, and wherein the distal end is secured to the first elongate, flexible member between the passage and the distal end of the first elongate, flexible member.

The second elongate, flexible member can enclose at least a portion of the first elongate, flexible member and the distal end of the second elongate, flexible member can be substantially co-extensive with the distal end of the first elongate, flexible member. The second elongate, flexible member can be inverted by pulling the first elongate, flexible member in a proximal direction. A thermally transmissive fluid can be provided for circulation through the lumens defined by the first and second elongate, flexible members and the passage defined by the wall portion of the first elongate, flexible member. As at least a portion of the device can be thermally conductive, thermal transfer between the fluid and a point exterior to the device can be effectuated.

The invention also includes a method for imparting a thermal change to a selected region within a skull. Steps of the method include creating an opening in the skull and inserting a thermal treatment device into the opening, the thermal treatment device having a first elongate, flexible member within a second elongate, flexible member. Next, the thermal treatment device is positioned proximate the selected region and a thermal transfer proximate the selected region is effected. Then, at least a portion of the second elongate, flexible member is inverted and the thermal treatment device is removed from the skull. In an exemplary method, the first elongate, flexible element is pulled through the opening, thereby inverting the second elongate, flexible member that is pulled from the opening after the first, elongate flexible element.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a side view of a neurosurgical device constructed in accordance with the principles of the present invention;

FIG. 2 is a side view of an exemplary fitting constructed in accordance with the principles of the present invention;

FIG. 3 is a section view taken along section 3-3 in FIG. 1;

FIG. 4 is a view of the device of FIG. 1 within a patient's skull;

FIG. 5 is another view the of the device within a patient's skull;

FIG. 6 is yet another view of the device within in a patient's skull;

FIG. 7 is a view showing the device of FIG. 1 during a removal step of a medical procedure;

FIG. 8 is a view showing the removal step of FIG. 7 within the skull;

FIG. 9 is another view of the device during a removal step;

FIG. 9A illustrates a completely inverted device;

FIG. 10 is a top view of the device configured as a spiral; and

FIG. 11 is a top view of the device configured as a pad.

### DETAILED DESCRIPTION OF THE INVENTION

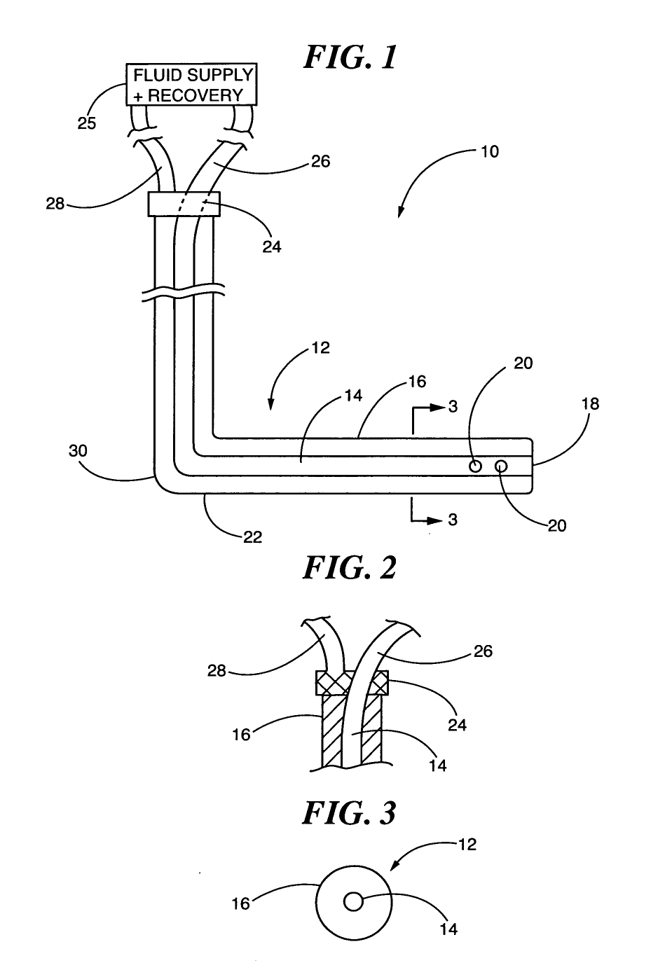

Referring now to the drawing figures in which like reference designators refer to like elements, there is shown in FIG. 1 a neurosurgical device constructed in accordance with the principles of the present invention and designated generally as 10. Device 10 includes an elongate, catheter-like element 12 that includes a first elongate member 14 defining a first lumen within a second elongate member 16 that defines a second lumen. Thus, the first and second lumens are generally coaxial. However, as used herein, the term “substantially coaxial” also encompasses an arrangement in which the first member 14 floats freely within the second member 16 or is anchored within the boundary of the second member. The elongate, catheter-like element 12 can be provided with a bend region 30 to provide what appears to be an “L” configuration. However, instead of (or in addition to) the illustrated bend, the device can assume shapes from linear to multiple complex curves at one or more points along its length.

The first member 14 and the second member 16 are crimped, closed or sealed at their distal ends. As shown in FIG. 1, the distal ends form or define the distal end 18 of the elongate element 12. Alternatively, the distal ends of the first and second members 14 and 16, respectively, can be provided with a sealing element, cap or wall element. Although FIG. 1 shows distal end 18 as being substantially flat or planar, it can also include a curved surface. One or more openings 20 are defined by the first member 14 at one or more points between the proximal and distal ends of the first member to create a flow path from first member lumen to the second member lumen. The first and second elongate members 14 and 16 can be substantially equivalent in length or the second elongate member can be shorter than the first elongate member, so long as it encloses the openings. As described in more detail below, the outer surface of the second elongate member 16 and the distal end 18 provide a tissue contact surface 22. The tissue contact surface 22 or other portions of second member 16 can provide an impermeable fluid barrier or, in other embodiments, a fluid-perfusive surface.

At the proximal end of the element 12, a cap or fitting 24 provides a fluid-tight seal and links a fluid supply and recovery system 25 to the element. The fluid supply and recovery system 25 provides a thermally-transmissive fluid, such as saline or a refrigerant which is cooled by a thermoelectric cooler or other known device for imparting a thermal change to a fluid. The fitting 24 fluidly couples a fluid input member 26 to the first member 14 and fluidly couples a fluid output member 28 to the second member 16. An exemplary side view of fitting 24 is shown with reference to FIG. 2. It is contemplated that first member 14 and fluid input member 26 can be coupled together and/or fabricated as a contiguous piece and that fitting 24 can define an opening such that first member 14 and fluid input member 26 can slidably engage and pass through the opening in a fluid-tight manner as is known in the art. Thus, the fitting 24 provides a fluid tight seal preventing cross flow between the first member 14 and the second member 16 while still allowing first member 14 to be able to be engaged or disengaged through fitting 24. Fitting 24 can be made of metal, plastic or any other polymer suitable for use in a medical environment.

FIG. 3 is a sectional view taken along section 3-3 in FIG. 1, wherein the second member 16 surrounds the first member 14. Of note, although the members are shown to be substantially cylindrical, the second member 16 can include one or more flat faces, or a non-uniform curvature to provide a selected contour to the tissue contact surface 22.

The first and second members 14 and 16 are constructed of one or more of compliant, non-compliant, and partially compliant polymers such as silicone polymer, PET, soft pellethane (such as pellethane 80AE or PEBAX 42). As described below in greater detail, the second member is constructed of a material that is sufficiently thin and flexible so as to be easily invertible. In an exemplary embodiment, first member 14 has an outer diameter of 0.055″ and a lumen wall thickness of 0.00025″. In an exemplary embodiment, second member 16 has an outer diameter of 0.085″ and a lumen wall thickness of 0.00025″.

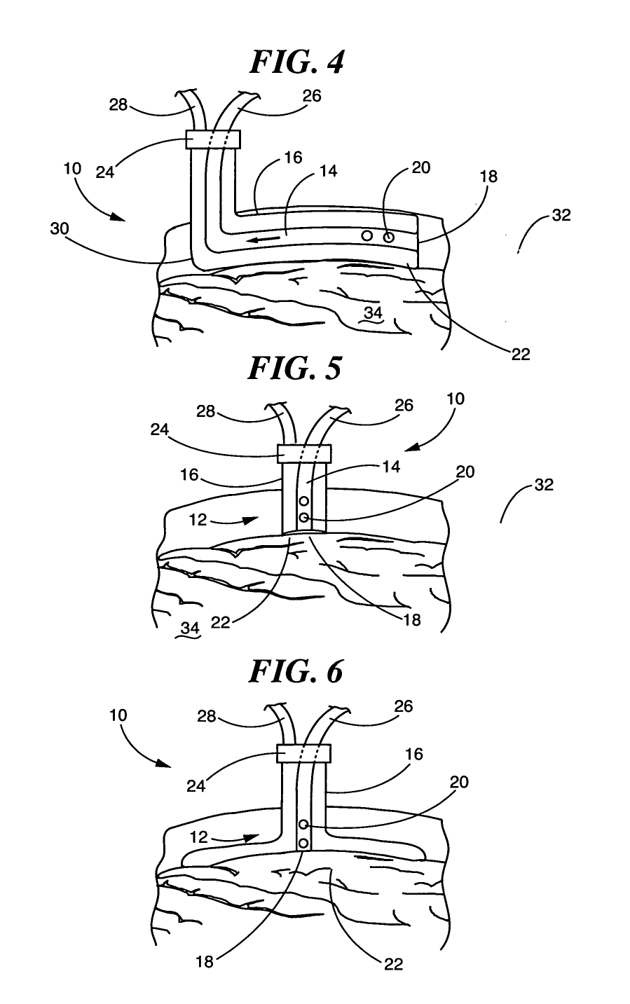

Turning now to FIG. 4, additional features of the device are described in conjunction with a method of use for cooling a selected area of tissue or fluid within the skull. As an initial step, a medical team creates an opening in the skull 32 using techniques known in the art, such as creating a burr hole. Known techniques are used to expose a desired layer of tissue 34 within the skull. A distal portion of the device is passed through the opening in the skull 32 and positioned at a selected location proximate the area to be treated. Refrigerant at a selected flow rate and temperature is directed through the first member 14 and evacuated from the second member 16 for a selected length of time. At the completion of the treatment, refrigerant flow is terminated and the distal portion of the device is withdrawn from the skull.

Although the device can be substantially shape retaining and impart thermal therapy, as shown in FIG. 4, the tissue contact surface 22 readily conforms to the tissue 34. This can be facilitated, for example, by introducing or evacuating fluid from the second member 16 a rate that does not create any, or sufficient, backpressure so as to inflate or harden the second member 16.

Whereas FIG. 4 illustrates tissue contact along a longitudinal “side” portion of the second member 16, FIG. 5 shows tissue contact using the distal end of device. As shown in FIG. 5, the second member 16 can be expanded to fill the opening in skull 32.

Referring now to FIG. 6, the distal portion of the second member can be expanded to a width that is significantly greater than the opening in skull 32 to provide additional tissue contact surface area. The expanded tissue contact surface 22 thus encompasses a large, substantially circular surface area. Modifications to the second member 16 can be made to provide a tissue contact surface 22 that has a different footprint.

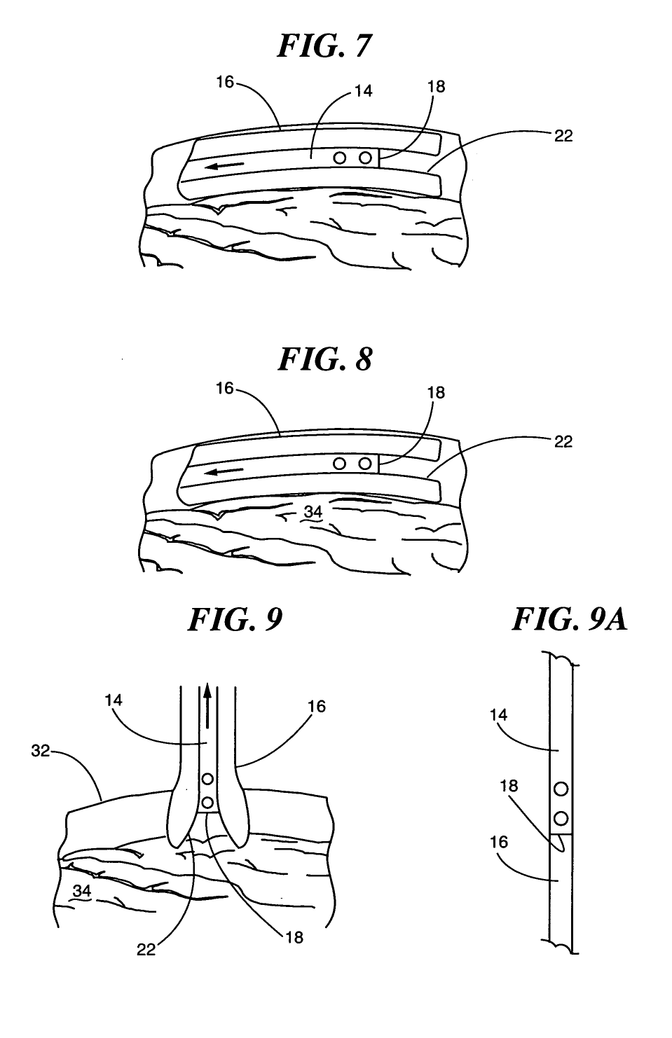

FIGS. 7-9 illustrate the device after the thermal treatment is complete and it is desired to withdraw or remove the distal portion of the device from the skull. In a first step, the thermally transmissive fluid is evacuated from first and second members 14 and 16. Traction is applied to the first member 14 in the direction of the arrow shown in the respective drawing figures causing the distal end of the first member to move proximally. Because the first member 14 is joined to the second member 16 near or at their respective distal ends, pulling the first member inverts the second member 16 and the distal end of the second member moves proximally while the proximal end of the second member moves distally until the second member is fully inverted as shown in FIG. 9A. This has the effect of rolling the second member 16 and the tissue contact surface 22 that it defines away from the treated tissue. This arrangement advantageously avoids dragging device 10 across the tissue and prevents tissue injury resulting from the removal of device 10.

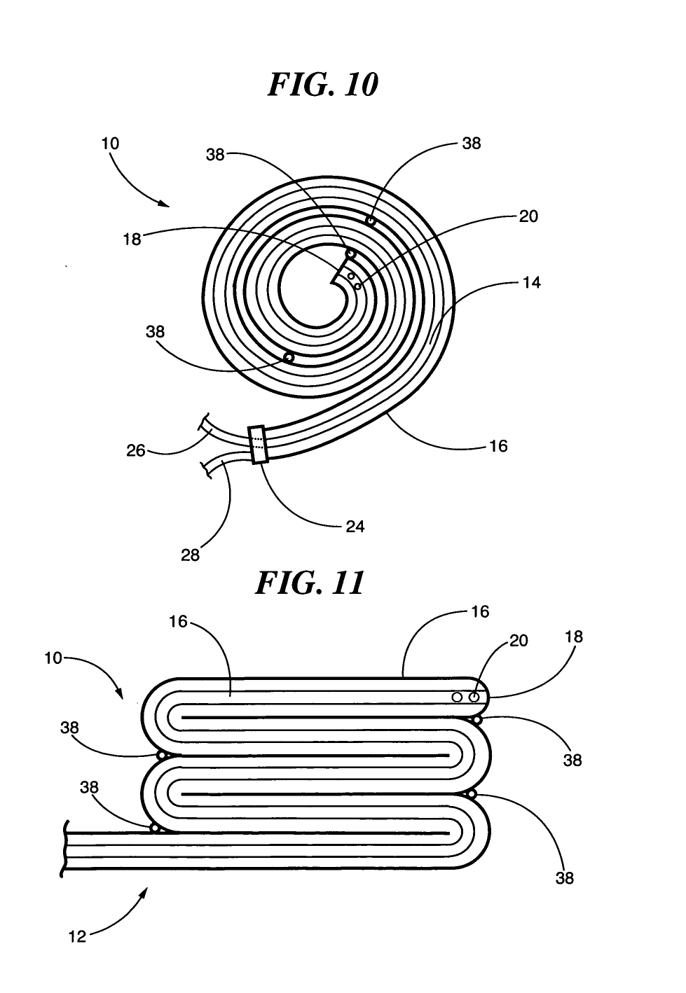

Referring now to FIGS. 10 and 11, the device is shown with its distal portions configured in a predetermined non-linear array. For example, FIG. 10 shows the device with a spiral pad configuration such that thermally transmissive fluid flows into the center of the device via first member 14 and toward the outer edges of the device via second member 16. To facilitate insertion, use and extraction, one or more coupling elements 38 are provided to maintain the spiral shape during insertion and use. Coupling elements 38 can be any adhesive that is strong enough to maintain the spiral shape during insertion and treatment, but which allow the adjacent coupled portions of second member 16 to uncouple from each other during extraction. In other words, when first member 14 is pulled proximally as described above with respect to FIGS. 7-9, the lateral and transverse forces created as a result are sufficient to cause coupling elements 38 to release their bonds with second member 16 to facilitate extraction of the device from the tissue treatment site.

FIG. 11 shows the device configured as a substantially rectangular pad. As with the embodiment shown in FIG. 10, coupling elements 38 are used to maintain the shape of the device during insertion and treatment, yet allow adjacent portions of second member 16 to decouple from one another during extraction.

The above-described device advantageously provides a solution for controlling the temperature of a localized regional brain tissue. Circulating a chilled fluid to lower the localized brain temperature serves as a neuro-protective means in a cerebral ischemia condition. It is also contemplated that the above-described devices can additionally be used to cool localized regions of the brain in a brain trauma patient as a way to lower cerebral metabolic requirements and minimize brain edema. Furthermore, the device can also be used in any post-operative trauma situation when the possibility of cerebral edema exists.

Although the above-described embodiments are discussed with respect to thermal treatment of the brain through the direct contact therewith, it is contemplated that the devices can be used in other procedures. For example, the devices can be placed through the nose into the ethmoid sinus to thermally treat carotid blood as it courses through the cavernous sinus up to the brain. Further, the device can be placed adjacent the hypothalamus and a warm fluid circulated through the device to raise the temperature perceived by the hypothalamus, thereby triggering peripheral vasodialation and systemic cooling.

The above-described device can also be used in other parts of the body when local tissue temperature control is desired. For example, the device could be applied to organs prior to or after transplant to minimize ischemia and swelling.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. A medical device comprising:
a first elongate, flexible member having a proximal end, a distal end, and a wall portion having an outer surface and an inner surface, the wall portion defining a lumen extending from the proximal end to the distal end, wherein the proximal end is open and the distal end is closed, and the wall portion further defining a passage from the outer surface to the inner surface at a point between the proximal end and the distal end;
a second elongate, flexible member having a proximal end, a distal end, and a wall portion having an outer surface and an inner surface, the wall portion defining a lumen extending from the proximal end to the distal end, wherein the proximal end is open and the distal end is closed, and wherein the distal end is secured to the first elongate, flexible member between the passage and the distal end of the first elongate, flexible member.

2. The device according to claim 1, wherein the second elongate, flexible member encloses at least a portion of the first elongate, flexible member.

3. The device according to claim 1, wherein the distal end of the second elongate, flexible member is co-extensive with the distal end of the first elongate, flexible member.

4. The device according to claim 1, further comprising a distal wall to which are attached the distal end of the second elongate, flexible member and the distal end of the first elongate, flexible member.

5. The device according to claim 4, wherein the distal wall is substantially planar.

6. The device according to claim 1, further comprising a second passage defined by the wall portion of the first elongate, flexible member from the outer surface to the inner surface at a point between its proximal end and the distal end.

7. The device according to claim 1, wherein the second elongate, flexible member is inverted by pulling the first elongate, flexible member in a proximal direction.

8. The device according to claim 1, wherein distal portions of the first and second elongate, flexible members are configured in a predetermined non-linear array.

9. The device according to claim 1, further comprising a thermally transmissive fluid, the thermally transmissive fluid transiting the lumens defined by the first and second elongate, flexible members and the passage defined by the wall portion of the first elongate, flexible member.

10. The device according to claim 9, further comprising a distal wall to which are attached the distal end of the second elongate, flexible member and the distal end of the first elongate, flexible member, and wherein the distal wall permits transfer of thermal energy between the thermally transmissive fluid and a point exterior to the device.

11. The device according to claim 6, wherein the second elongate, flexible member permits transfer of thermal energy between the thermally transmissive fluid and a point exterior to the device.

12. The device according to claim 9, wherein introduction of the thermally transmissive fluid into the first and second elongate, flexible members causes them to expand.

13. A medical device comprising:
a first elongate, flexible member having a proximal end, a distal end, and a wall portion having an outer surface and an inner surface, the wall portion defining a lumen extending from the proximal end to the distal end, wherein the proximal end is open and the distal end is closed, and the wall portion further defining a passage from the outer surface to the inner surface at a point between the proximal end and the distal end;
a second elongate, flexible member having a proximal end, a distal end, and a wall portion having an outer surface and an inner surface, the wall portion defining a lumen extending from the proximal end to the distal end, wherein the proximal end is open and the distal end is closed, and wherein the distal end is secured to the first elongate, flexible member between the passage and the distal end of the first elongate, flexible member,
wherein the second elongate, flexible member encloses at least a portion of the first elongate, flexible member,
wherein the distal end of the second elongate, flexible member is substantially co-extensive with the distal end of the first elongate, flexible member; and
wherein the second elongate, flexible member is inverted by pulling the first elongate, flexible member in a proximal direction.

14. The device according to claim 13, further comprising a thermally transmissive fluid, the thermally transmissive fluid transiting the lumens defined by the first and second elongate, flexible members and the passage defined by the wall portion of the first elongate, flexible member.

15. The device according to claim 14, further comprising a distal wall to which are attached the distal end of the second elongate, flexible member and the distal end of the first elongate, flexible member, and wherein the distal wall permits transfer of thermal energy between the thermally transmissive fluid and a point exterior to the device.

16. The device according to claim 15, wherein the second elongate, flexible member permits transfer of thermal energy between the thermally transmissive fluid and a point exterior to the device.

17. The device according to claim 16, wherein introduction of the thermally transmissive fluid into the first and second elongate, flexible members causes them to expand.

18. A method for imparting a thermal change to a selection region within a skull comprising the steps of:
creating an opening in the skull;
inserting a thermal treatment device into the opening, the thermal treatment device having a first elongate, flexible member within a second elongate, flexible member;
positioning the thermal treatment device proximate the selected region;
effecting a thermal transfer proximate the selected region;
inverting at least a portion of the second elongate, flexible member; and
removing the thermal treatment device from the skull.

19. The method of claim 18, wherein the steps of inverting and removing include the step of pulling the first elongate, flexible element through the opening, thereby inverting the second elongate, flexible member which is pulled from the opening after the first, elongate flexible element.
